(19) United States
(12) Patent Application Publication
(10) Pub. No.: US 2019/0302110 A1
(43) Pub. Date: Oct. 3, 2019

(54) Quick validation method for binding kinetic analysis

(71) Applicant: Biosensing Instrument Inc., Tempe, AZ (US)

(72) Inventors: Nguyen Ly, Tempe, AZ (US); Tianwei Jing, Tempe, AZ (US); Feimeng Zhou, Temple City, CA (US)

(21) Appl. No.: 15/910,840

(22) Filed: Mar. 2, 2018

Related U.S. Application Data
(60) Provisional application No. 62/465,954, filed on Mar. 2, 2017.

(51) Int. Cl. G01N 33/557; G01N 21/552

(52) U.S. Cl. CPC G01N 33/557; G01N 21/553

(57) ABSTRACT

A novel validation method for binding kinetic analysis with a surface plasmonic apparatus is disclosed. The method includes a new approach to quickly validate the fidelity of the measured data and facilitate an accurate binding kinetic analysis of biomolecular interaction. It utilizes multiple sensing surface areas to immobilize different amounts of a ligand, followed by an injection of analyte solution through either all of the sensing surface areas or predetermined zones. The binding data between analyte and ligand are checked against pseudo-first order binding kinetics of bimolecular reactions, and, with the proper validation of the data, the binding kinetics of the interaction can be determined with high degree of accuracy without many measurements of other analyte concentrations and repeated regeneration of the sensor surface.

### CROSS-REFERENCE TO RELATED APPLICATION

This application claims the benefit of United States Provisional Application No. 62/465,954 filed on Mar. 2, 2017

### REFERENCES CITED

1. Homola, J., (2003) Present and future of surface plasmon resonance biosensors. Anal Bioanal Chem, 377(3): p. 528-39.
2. Schuck, P., & Zhao, H. (2010). The role of mass transport limitation and surface heterogeneity in the biophysical characterization of macromolecular binding processes by SPR biosensing. Methods Mol Biol, 627, 15-54.
3. Karlsson, Robert, Anne Michaelsson, and Lars Mattsson. “Kinetic analysis of monoclonal antibody-antigen interactions with a new biosensor based analytical system.” Journal of immunological methods 145.1-2 (1991): 229-240.
4. Myszka, D. G. (1999). Improving biosensor analysis. Journal of Molecular Recognition, 12(5), 279-284.
5. Karlsson, R. Roos, H; Fagerstam, L; Persson, B. (1994). Kinetic and Concentration Analysis Using BIA Technology. Methods: A Companion to Methods in Enzymology, 6, 99-110.
6. Khalifa, M. B.; Choulier, L.; Lortat-Jacob, H.; Altschuh, D.; Vernet, T. Anal. Biochem. 2001, 293, 194-203.
7. Morton, T. A.; Myszka, D. G.; Chaiken, I. M. Anal. Biochem. 1995, 227, 176-85.

### FIELD OF THE INVENTION

The present invention is related to applications and methods which utilize surface plasmonic (SP) absorption at or near its resonance for detecting the presence and absence of molecules, biological cells or organelles on or near a surface, or structural and electronic changes of the molecules through their interactions with an analyte, particularly to a method of characterizing interactions between the analyte in the fluid and the ligand immobilized onto multiple sensing surface areas.

### BACKGROUND OF THE INVENTION

Many analytical techniques are used to characterize interactions between biomolecules. For example, the protein-drug interaction is an important type that is of fundamental importance to molecular biology, immunology, drug discovery, and pharmacokinetics and pharmacology. In this context, the analytical techniques focus on immobilization of a ligand, such as an antibody or other biomolecules, to a sensing surface, followed by exposing the ligand with an analyte, such as an antigen, via a fluidic delivering system. With exposure of the ligand to analyte, some characteristic change of the sensing surface is measured, and the change is indicative of the interaction, such as the ability of the ligand to bind the analyte and the rate of binding.

The Surface Plasmon Resonance (SPR) sensor system is a particularly popular method to monitor biomolecular interactions in real time without the necessity of attaching a label to the analyte. Many commercial biosensor systems based on SPR have become powerful tools for such interaction analysis. A more detailed description of SPR and theory of detection may be referred to [Reference 1]. All SPR systems use a flow system for sample delivery and optoelectronic elements to detect the analyte-ligand interaction at the sensing surface. By measuring the changes of the SPR angle, a plot (sensorgram) of the response signal vs. time is generated and analyzed to obtain relevant information on binding interactions, such as affinity and association and dissociation rates of the ligand-analyte binding complex.

A representative sensorgram for a reversible interaction at the sensing surface is presented in

FIG. 1, the sensing surface 1 having an immobilized ligand (capturing molecule), e.g. an antibody, interacting with analyte 2, e.g. an antigen presents in the injected sample. The Y-axis indicates the response (measured signal due to SPR angle changes) and X-axis indicates the time. Before the analyte is introduced onto the sensing surface pre-immobilized with a ligand, a constant flow of analyte-free running buffer is maintained to stabilize the baseline 3 (i.e. background). When the analyte in a sample is injected into the flow stream, it flows over the sensing surface and gets exposed to the ligand pre-immobilized onto the sensing surface, where the binding occurs. This is referred to as the association phase. The binding events is detected as the SPR response signal 4 rising as a function of time t (in the form of 1−e^{-t} at wherein a is a constant).

After the sample injection is completed, the sensing surface is immediately flushed with an analyte-free buffer to allow dissociation of the analyte from the binding complex to take place, this is referred to as the dissociation phase, which causes the response signal 5 to decay as a function of time (in the form of e^{-kd t} wherein k_{d} is the dissociation constant). Furthermore, the sensing surface can be “washed” as shown in step 6 with an agent to “regenerate” the original surface without any analyte or even the pre-immobilized ligand, thereby allowing the sensor surface 1 to be reused for subsequent measurements. In practice, most sensor chips can only be “reused” for a limited time (generally less than 10 cycles) and in many cases the sensing surface cannot be regenerated at all. This aspect of SPR measurements significantly affects both the sample throughput and increases the assay costs and has not been sufficiently addressed in the past.

The sensorgram provides the essential information from which the binding kinetic data could be readily derived using pseudo-first order binding kinetics theory. This protocol has been widely used in all SPR biosensing measurement for the last 20 years. However, despite the development of highly advanced fluidic systems and sophisticated modification of sensor surface, as well as numerous publications on various applications of SPR to a wide range of biomolecules, optimization of SPR measurement parameters remain difficult and requires good knowledge of surface chemistry, mass transfer limitation, kinetic theory, and etc. [Reference 2-3]. Mistakes are often made with inaccurate kinetic data, due to the lack of understanding on how these secondary effects (mentioned above) impact the observed binding behaviors. To improve the quality and accuracy of the binding kinetic measurement, a wide range of concentrations of the analyte with numerous repeats and carefully designed references must be used to identify and eliminate the secondary effects in the measurement. [Reference 4]

In a typical SPR experiment on the “1:1” reaction, the ligand B is immobilized onto a sensor surface and its interaction with an analyte A injected onto the sensing surface through a microfluidic channel can be expressed as:

$$A + {B}_{{←}_{{k}_{d}}}^{{\to }^{{k}_{a}}} AB \qquad ( 1 )$$

During the association phase (rate constant ka), the SPR signal (Ron) is given by

$${R}_{on} = \frac{{k}_{a} {CR}_{max}}{{k}_{a} C + {k}_{d}} ( 1 - {e}^{- ( {k}_{a} C + {k}_{d} ) t} ) \qquad ( 2 )$$

where C is the analyte concentration, kₐ the association rate constant, Rₘₐₓ the maximum analyte coverage at the sensor, and t the reaction time.

To initiate the dissociation reaction, the injected analyte is rapidly replenished with a running buffer. The SPR signal (R_{off}) decays exponentially with time from R₀, the signal at the moment when buffer replaces the analyte in the fluidic channel:

R_{off}=R₀e^{-k}^{_{d}}^{t} (3)

If sufficient time is allowed during the sample injection for eq. 1 to reach the equilibrium, the SPR signal reaches a steady-state value R_{eq}, as shown by eq. 4:

$${R}_{eq} = \frac{{k}_{a} C}{{k}_{a} C + {k}_{d}} {R}_{max} \qquad ( 4 )$$

To derive kinetic quantities of ka and kd, and thermodynamic parameters KD=kd/ka and Rmax, either several Rₘₐₓ or C values in eq. 2 are needed for reliable fitting of the association phase as eq. 2 has multiple variables. Traditionally, a series of analyte concentrations have to be injected to generate many curves of Rₒₙ(t), which are subsequently fitted with the kinetic theory. Notice in eq.4, R_{eq} vs C should follow the Langmuir isotherm behavior and KD=kd/ka can be derived without knowing the ka and kd values. For example, as shown in FIG. 2A and FIG. 2B, Seven different concentration of analyte is used to measure the binding behavior (FIG. 2A) and the R_{eq} for each concentration C are obtained labeled 21 to 27 and plotted against its concentration in log scale (FIG. 2B). Based on eq. 4, when Req=½ Rmax, C=affinity KD .

So far, there is no attempt on varying Rₘₐₓ at a fixed C value in eq. 2 to calculate kinetic quantities. To vary Rₘₐₓ and fix C, it will require an SPR system which has at minimum of 3 channels to immobilize the ligand with different and known surface densities.

It is important to note that eq. 1 assumes that the reaction has a pseudo-first order binding kinetic behavior, ignoring all the secondary effect such as analyte aggregation, nonspecific absorption, bulk refractive index change, mass transport limitation and etc. When secondary effects are considered, the kinetic behavior of eq.2 and eq.4 become

$$\begin{matrix}{R}_{on} = \frac{{k}_{f} {CR}_{max}}{{k}_{f} C + {k}_{r}} ( 1 - {e}^{- ( {k}_{f} C + {k}_{r} ) t} ) and & ( 5 ) \\ {R}_{eq} = \frac{{k}_{f} C}{{k}_{f} C + {k}_{r}} {R}_{max} & ( 6 )\end{matrix}$$

respectively. In eqs. 5 and 6. k_{f} and kᵣ represent the forward association and reverse dissociation rate constants. They are functions of k_{a,} k_{b,} the amount of ligand immobilized [B], and the rate of analyte mass transport to the surface (kₘ). In addition, bulk refractive index change will lead to large baseline variations, and secondary effects will cause complicated binding behavior to change. Therefore, the pseudo-first order binding kinetic theory may become invalid. In this case, R_{eq} in eq.6 at equilibrium state is no longer directly proportional with R_{max.} It has been extremely difficult to calculate kinetic quantities of kₐ and k_{d} accurately in the presence of the secondary effects.

There is only one exception in the secondary effects which the pseudo-first order binding kinetic theory can still be used with minor corrections, that is when the binding event is partially controlled or limited by the mass transport (MT). Assuming MT is the only secondary effect present, k_{f} and kᵣ can be represented as:

$$\begin{matrix}{k}_{f} = {k}_{a} \frac{{k}_{m}}{{k}_{m} + {k}_{a} [ B ]} & ( 7 ) \\ {k}_{r} = {k}_{d} \frac{{k}_{m}}{{k}_{m} + {k}_{a} [ B ]} & ( 8 )\end{matrix}$$

By substituting eq.7 and eq.8 into eq. 6, R_{eq} becomes the same as eq.4. This implies that in the presence of the MT effect, the pseudo-first order binding kinetic theory with modified k_{f} and kᵣ can still describe the binding kinetic behavior [Reference 5].

Various practices for minimizing the secondary effects have been exercised over the past 15 years, which include: using the ligand density as low as possible to avoid complete or partial MT limitation; and for high-affinity binding involving small analyte molecules, using a high immobilized ligand density as the steric hindrance is small for small molecules that diffuse much faster than bulky biomolecules. To optimize the measurement condition and to minimize the secondary effects, injections of various analyte concentrations with regeneration cycles in between have to be conducted to validate the measurement and fitted with the pseudo-first order kinetics. Often the Langmuir isotherm curve is used to fit with R_{eq} vs C in order to validate the data quality. This brutal force approach is necessary because there is not a simple way to validate the measured data suitable for accurate pseudo-first order binding kinetic fit when different analyte concentration C are used. [Reference 6-7]

This widely accepted approach (i.e. fixing the ligand density and varying the analyte concentrations) is tedious and costly in terms of sample consumption and resources (analysis time, labor, reagents, sensor chips, etc.). A major problem inherent in the conventional approach is that the sensing surfaces must be “refreshed” or “regenerated”, which also degrades sensing surface and leads to error in measurements, and in some cases, is not possible.

Another problem is that the concentration of the analyte directly affects the time scale for binding kinetic to reach equilibrium as shown in equation 2. When a lower analyte concentration is used, the time takes to reach equilibrium state increases as the exponential coefficient in eq. 2 becomes smaller. This prolongs the measurement time significantly.

It is the principal objective of the present invention to overcome the above mentioned problems in the prior arts by providing a novel method to validate the binding data suitable for pseudo-first order binding kinetic fit in measuring the said interaction of an analyte with ligands, therefore, kinetic parameters can be quickly and accurately determined without making injections of multiple solutions and surface regeneration.

### SUMMARY OF THE INVENTION

In the present invention, by focusing on varying the immobilization amount of a ligand (Rmax) instead of the analyte concentration (C) in eq. 2, a novel method is provided for validating the kinetic data in measuring the interaction of an analyte in the flowing stream with a ligand immobilized on the sensing surfaces. First, using a multi-channel SPR instrument to prepare multiple sensing areas through an in-line process to controllably immobilize graduated amounts of a ligand onto each area or zone. Then for binding kinetic measurements, an injection of analyte of a given concentration is made to flow over the said areas to obtain its binding kinetics. To validate that the measurement obeys the pseudo-first order binding kinetics, a plot of equilibrium binding responses (R_{eq}) vs. the amounts of immobilized ligand (R_{immob}, which is propotional to Rₘₐₓ) is generated. Only when R_{eq} is directly proportional to R_{immob} will the pseudo-first order condition be valid. If a portion of the plot is not directly proportional to R_{immob} due to the secondary effects, it will be discarded. In other words, only the data in the region wherein a proportional relationship exists will be used for calculating the binding kinetics.

As a result, the new approach removes many uncertainties without injection of multiple solutions and surface regeneration. Consequently, the method saves time, samples, and sensor chips, and allows equilibria to be established at the same time, and avoids any damages/changes that may occur at the sensor surface during its regeneration. It is worth noting that even though in theory only three channels are needed (two channels necessary to have two signals for solving two unknown ka and Rmax, and one channel for background signal recording), in practice five or more channels will be essential to exclude the interference caused by the secondary effects). This is because any two data points (two signals from two channels after subtraction of the background from the third channel) can render a liner plot. Only with at least 3 data points can a linear regression be meaningful. With four or more data points (i.e. five or six fluidic channels), the fidelity of the linear regression is further enhanced.

In one aspect, the present method is carried out by controlling the exposure (immobilization) time of the ligand to the sensing surface in each channel, so various ligand amounts can be immobilized. Afterward, loosely bound ligand molecules are washed off and the active or unreacted sites remaining on the sensor chip are blocked. The responses of the binding kinetics of ligand with analyte are measured by a single injection of the analyte into the flowing stream over the multiple sensing surface areas.

In another aspect, the present method keeps a fixed concentration and varies immobilization amount R_{immob}. according to equation 2, all exposed sensing areas or channels reach equilibria R_{eq} at the same time because (kₐC+k_{d} )t is the same for all sensing areas. This allows quick affinity measurement to be carried out by using a simple linear regression fit of the data to

$${R}_{eq} = \frac{C}{C + {K}_{D}} {R}_{immob}$$

or eq 4 to obtain K_{D}.

In still another aspect, the present invention provides an analytical method for studying molecular interactions, which comprises a computer processing procedure including program code and algorithm for performing the steps of said method.

These and other aspects of the invention will be evident upon reference to the accompanying drawings and the following detailed description.

### BRIEF DESCRIPTION OF DRAWINGS

FIG. 1 is a sensorgram showing detector response versus time at different stages of the interaction between an analyte and an immobilized ligand on a sensor surface.

FIG. 2A is a set of sensorgrams obtained with seven different concentrations of the analyte injected onto a sensing surface with a fixed ligand density (the traditional method) with surface regeneration carried in between.

FIG. 2B is a plot of R_{eq} values vs. the analyte concentration, along with the curve fitting using the Langmuir isotherm. When R_{eq}=½ Rₘₐₓ, C=KD affinity

FIG. 3A illustrates four sensing areas (channels) immobilized with graduated amounts of a ligand from areas (channels) 31 to 34.

FIG. 3B is a set of measured sensorgrams during the immobilization process. ΔR_{immob} represents the immobilized amount at corresponding sensing areas 31 to 34.

FIG. 4A is a set of measured sensorgrams after an injection of an analyte onto multiple sensing areas via multiple channels.

FIG. 4B is a plot of R_{eq} vs Rₘₐₓ shows a directly proportionality.

FIG. 4C is a plot of R_{eq} vs Rₘₐₓ showing the deviation of the direct proportionality blue line due to secondary effects.

FIG. 5A illustrates sensorgrams obtained upon injections of 100 nM anti-GFP into channels pre-immobilized with GFP before and after regeneration by HCI pH=4.0, and NaOH pH=12.0.

FIG. 5B illustrates fluorescence spectra of 50 nM GFP before and after the addition of HCI pH 4.0 or NaOH pH=12.0 .

FIG. 5C illustrates sensorgrams of a single injection of 100 nM anti-GFP binding to pre-immobilized GFP at densities of 0.355, 0.571, 0.783, and 1.127ng/mm².

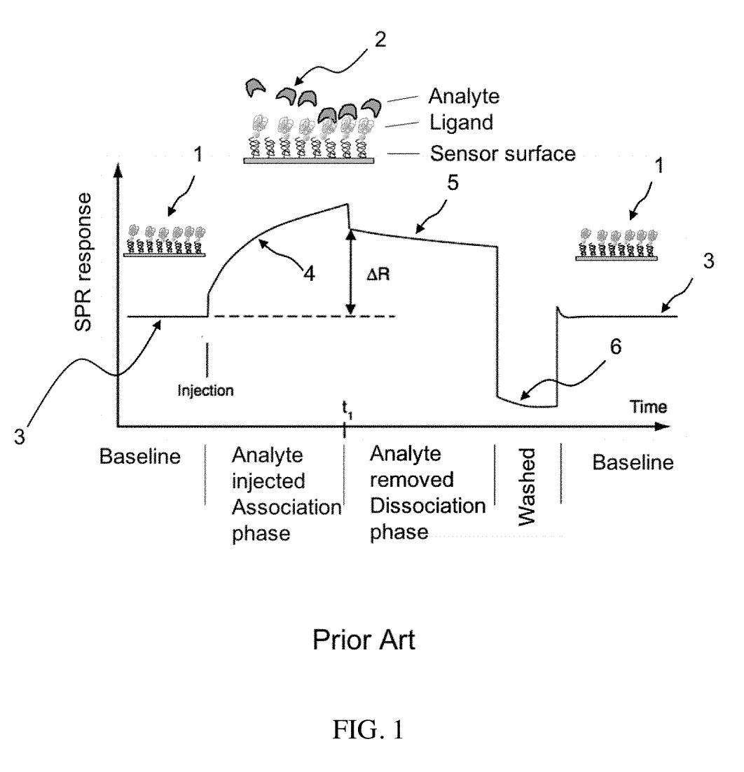

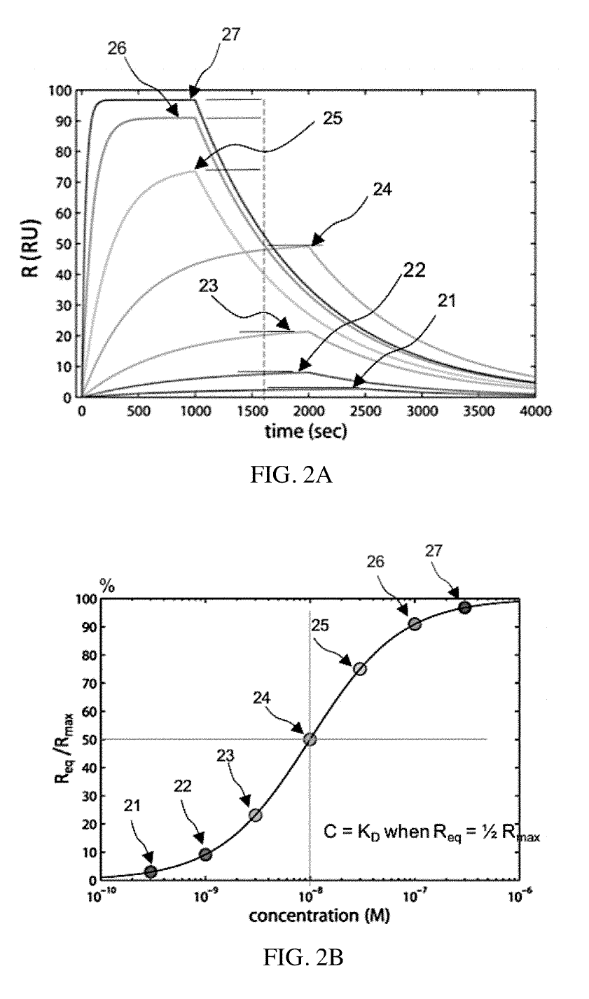

For simplicity and clarity of illustration, the drawing figures illustrate the general manner of construction, and descriptions and details of well-known features and techniques may be omitted to avoid unnecessarily obscuring and invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements.

### DESCRIPTION OF THE PROFFERED EMBODIMENT

As mentioned above, the present invention relates to a method for quickly validating the binding kinetic measurement for first-order binding kinetic region in assays or studies involving the detection of binding events of analyte at a sensing surface covered with immobilized ligand.

All measurements for binding kinetics in prior arts have been performed by measuring many binding curves with multiple injections of different analyte concentrations on the sensing surface with a fixed ligand density. With the availability of three or more channels in SPR systems such as the BI-4500 five-channel SPR system, it becomes possible to controllably vary immobilized ligand amounts instead of analyte concentration to perform kinetic measurements, thus providing many distinct advantages over prior art to validate the binding kinetic data based on the pseudo-first order binding kinetic theory.

In the following description of present art, various aspects of the present invention are disclosed more specifically for the purposes of illustration and not limitation.

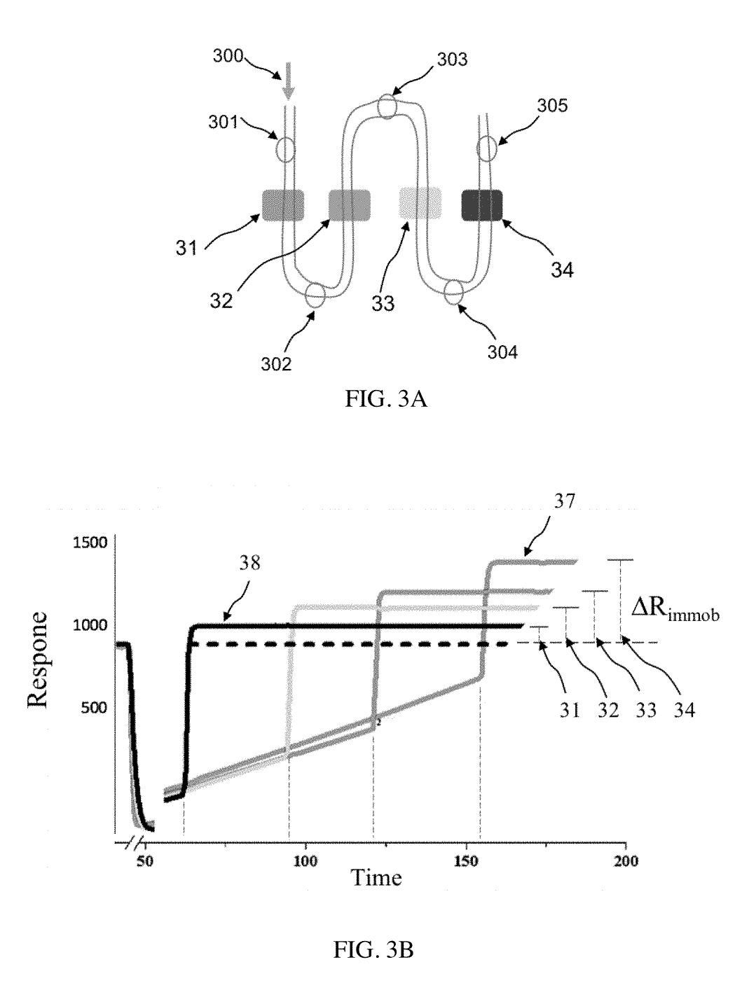

In the present invention, with a multi-channel SPR system and a single injection of only one ligand concentration, multiple sensing surface areas with different amounts (densities) of a ligand can be immobilized by simply exposing the ligand solution to each sensing area for different periods of time. As shown in FIG. 3A, four sensing area 31 to 34 are exposed to a flow stream that contains ligand in a serial manner, injecting from inlet 300 and flow out at 305. With a controlled timing, 304 opens to drain the stream and stop sensing area 34 to be exposed to ligand, and sequentially, 303 opens to stop exposing 33 area, and 302 opens to stop exposing 32 area, finally 301 opens to stop exposing 31 area. FIG. 3B shows the measured amount of ligand immobilized onto each sensing area (31 to 34), as the longer exposed time with ligand stream, the more ligand immobilized onto the area. the sensorgram curve 37 indicates the biggest ΔRimmob amount corresponding to 31 has the longest exposed time, and the sensorgram curve 38 represents the smallest ΔRimmob amount corresponding to 34 has the shortest exposed time. The capability of controlling the opening and closure of each individual channel facilitates the single injection and yields the desired immobilization densities.

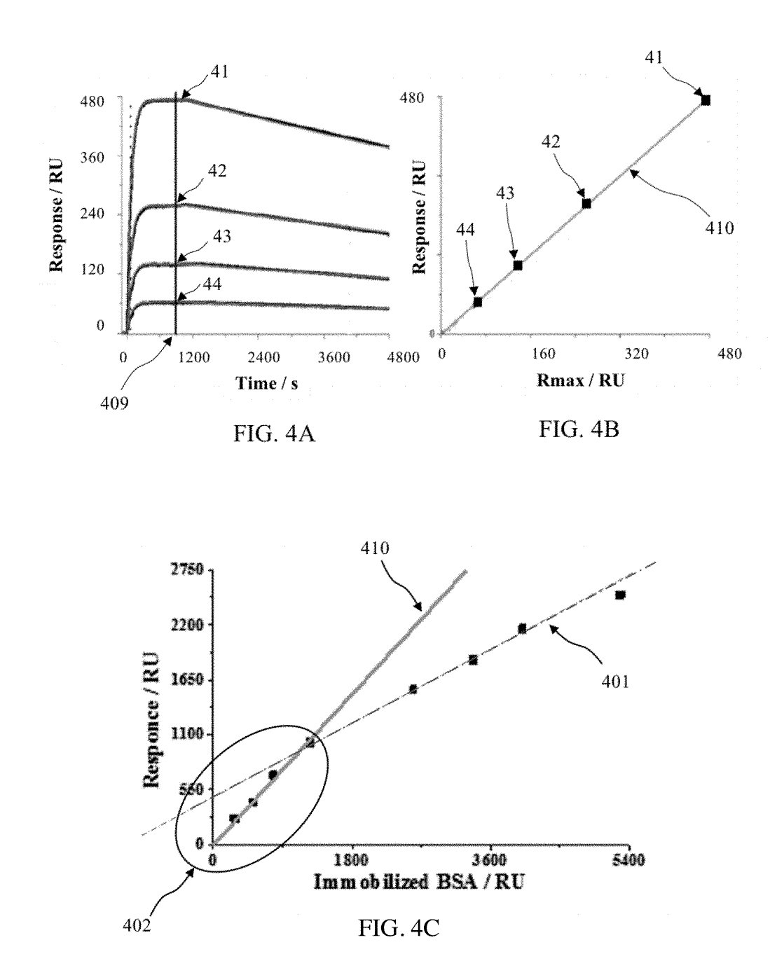

To perform binding kinetic measurements, an analyte of a given concentration is then injected into the flowing stream over the immobilized sensing areas, again in a single injection. The responses of binding kinetics are measured simultaneously from the sensing areas. FIG. 4A shows the sensorgram of four sensing areas with different immobilization amount of ligand. According to the pseudo-first order binding kinetics theory discussed above, eq. 4 predicts that the response (R_{eq}) from the analyte/ligand binding at equilibrium at a given concentration is directly proportional to the maximum SPR response (Rₘₐₓ) at the sensing area. Thus R_{eq} is also directly proportional to the amounts of the immobilized ligand (R_{immob}) since Rₘₐₓ is directly proportional to R_{immob}.

R_{eq}∝R_{immob}

In FIG. 4A the R_{eq} is measured at the intercept of line 409 with each sensorgram curve 41 to 44, and FIG. 4B shows the plot 410 of four Req vs Rimmob as directly proportional. If there are secondary effects, Req will not be directly proportional to the immobilized ligand density and will be trending down 401 due to the inefficiency of the binding in FIG. 4C. Therefore, eq.4 provides a quick validation for whether pseudo-first order binding kinetics theory can be used to obtain ka and kd. In contrast, the Langmuir isotherm curve fitting in the prior art using fixed Rₘₐₓ and varying C is more prone to errors as it is not obvious to judge for a good fit (due to the scaling problem inherent in plotting responses vs. the logarithm of the analyte concentrations.

In a variant of the present invention, eq.4 can also be used to find the right range of immobilized ligand densities that can be used to fit with pseudo-first order binding kinetic theory. As shown in FIG. 4C, only the framed region 402 near origin has the direct proportionality. This is because not only a good linearity was obtained, but also the resultant line goes through the origin. Therefore, the data in this region will yield accurate binding kinetic parameters by fitting with pseudo-first order binding kinetic theory. With this quick validation method, the need of multiple injections of analyte of various concentrations and regeneration of the sensor surface can be obviated. The new method facilitates kinetic measurements and saves time, samples and sensor chips.

In another variant of the present invention, it is evident that accurate binding kinetic data can be obtained and validated with a single injection of analyte into multiple channels with various immobilized ligand densities. This makes it possible to measure the interaction with certain type of ligand molecules or antigen that could not be regenerated.

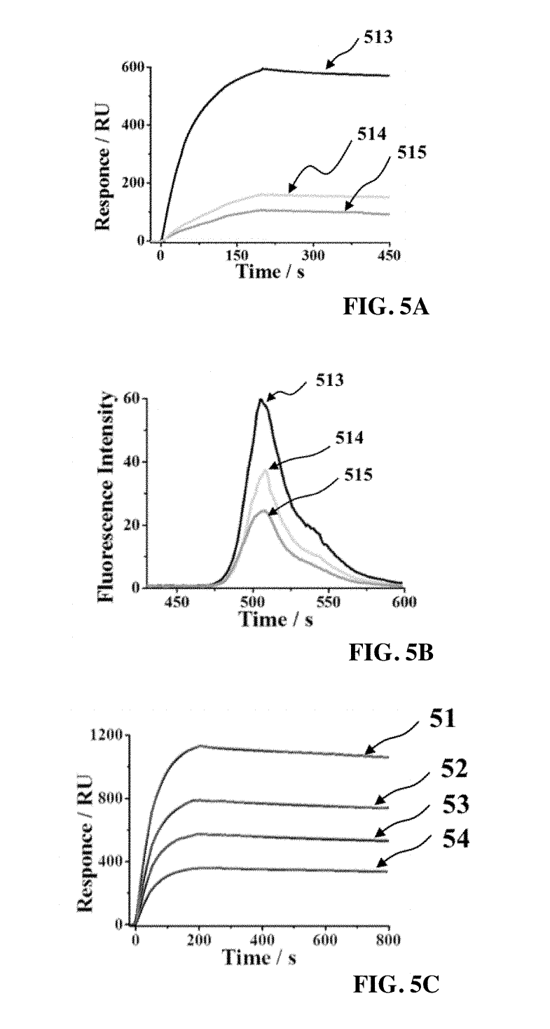

An example of binding kinetic measurement that is severely affected by the regeneration step is the interaction between a monoclonal antibody to the green fluorescent protein (GFP). GFP antibody is immobilized onto a sensor surface for binding kinetic measurement, but it cannot be regenerated because GFP would be greatly denatured by regeneration chemicals such as HCI or NaOH. Thus kinetic measurements of the interaction with injections of various analyte concentrations with regenerations in between could not be used in this case. FIG. 5A shows the effects of three regenerations to the GFP with anti-GFP binding sensorgram, with each regeneration, the GFP is greatly denatured resulting a much less response in binding measurement from curve 513 to curve 515. This denaturing is also confirmed by fluorescence spectra shown in FIG. 5B, as 50nM GFP before addition of HCI or NaOH shows highest response peak 513, as HCI added the peak starts to diminish 514 and with NaOH added the peak 515 goes much smaller.

Such a problem is mitigated with the present invention. With a graduated immobilization of GFP onto four sensing areas (channels) 31 to 34 as shown in FIG. 3A and a single injection of a relatively high analyte (anti-GFP) concentration, FIG. 5C shows a single injection of 100 nM anti-GFP binding to pre-immobilized GFP at densities of 0.355(54), 0.571(53), 0.783(52), and 1.127(51) ng/mm². The four sensorgram 51 to 54 corresponding to sensing area 31 to 34 with R_{eq} values can be simultaneously obtained without surface regeneration shown. Thus the binding kinetics can be measured. Such a procedure can be easily programmed, reducing the cost associated with sensor chips and reagents and simplifying the experiment geared towards affinity and kinetic measurements.

In still another variant of the present invention, using a fixed concentration and varying immobilization amounts provides a quick and simple way to obtain affinity K_{D} by using a simple linear regression fit with the said data set to equation 4 or R_{eq}=C/C+K_{D}R_{immob}. According to eq. 2, at fixed C, all exposed sensing areas (channels) reach quasi equilibrium R_{eq} at the same time as e^{-(k}^{C+k}^{_{d}}^{)t} is the same for all sensing areas. With a higher concentration can be used, the binding process will reach its equilibrium state at a faster rate, and with better signal-to-noise ratio. These factors all help improve the data quality. Comparing with conventional method of varying the analyte concentration C, the equilibrium establishment is dependent on C according to eq. 2. With higher C reaching the equilibrium faster than the lower ones, It makes the affinity measurements much more difficult to program and automated.

Although the invention has been described with reference to specific embodiments, it will be understood by those skilled in the art that various changes may be made without departing from the spirit or scope of the invention, accordingly, the disclosure of embodiments of the invention is intended to be illustrative of the scope of the invention and is not intended to be limiting. It is intended that the scope of the invention shall be limited only to the extent required by the appended claims.

What is claimed is:

1. A method for measuring and characterizing interaction of an analyte with a ligand immobilized onto a sensing surface, which comprises
a. A multi-channel SPR device equipped with a microfluidic system to immobilize a ligand onto multiple sensing areas of a sensor surface with controllable and gradually increased amounts and to deliver an analyte with predefined concentration onto the said areas.
b. A method to validate the measured data with a directly proportional relationship between the binding response (Req) of the analyte-ligand at a given analyte concentration at or near equilibrium and the maximum responses (Rmax) when all available immobilized ligand is bound at multiple sensing areas.
c. Follow-up pseudo-first order binding kinetics analysis to determine ka, kd, KD using the validated data sets.

2. In the method according to claim 1, the multi-channel SPR device is an apparatus that includes a sensor, a light source, optical assemblies, optoelectronic position detectors and electronics that is interfaced to a computer to determine the amounts of ligand immobilized, to control the analyte delivery, and to collect signals from the detectors for measurements. The sensor consists of a metal film and dielectric blocks which support the metal film as the sensing surface. The apparatus also includes a microfluidic module mounted on the sensor. The module has multiple inlet and outlet ports to control the fluid that flows over the sensing surface areas.

3. The method contains, according to claim 1, a sensor surface that may be modified by adding a layer of polymer, or other biomolecules.

4. The method contains, according to claim 1, Rₘₐₓ can be substituted with immobilized amounts (R_{immob}) of the ligand on the sensing area.

5. The method contains, according to claim 1, R_{eq} can be substituted with binding response R(t) at a given time t during the binding process.

6. The method contains, according to claim 1, analyte and ligand can be any atoms, ions, molecules, proteins, antibodies and antigens, biological cells or organelles.

7. A method for measuring and characterizing interaction of an analyte with a ligand immobilized onto a sensing surface, which comprises
a. A multi-channel SPR device equipped with a microfluidic system to immobilize a ligand onto multiple sensing areas of a sensor surface with controllable and gradually increased amounts and to deliver an analyte with predefined concentration onto the said areas.
b. A method to use a higher concentration (C) closer or larger than KD of an analyte, allowing binding to reach equilibria rapidly and simultaneously at all sensing areas.
c. A follow-up linear regression fit to R=_{eq}=C/C+K_{D}R_{immob} to validate the measured data sets and to determine affinity KD using the data sets.

8. In the method according to claim 7, the multi-channel SPR device is an apparatus that includes a sensor, a light source, optical assemblies, optoelectronic position detectors and electronics that is interfaced to a computer to determine the amounts of ligand immobilized, to control the analyte delivery, and to collect signals from the detectors for measurements. The sensor consists of a metal film and dielectric blocks which support the metal film as the sensing surface. The apparatus also includes a microfluidic module mounted on the sensor. The module has multiple inlet and outlet ports to control the fluid that flows over the sensing surface areas.

9. The method contains, according to claim 7, a sensor surface that may be modified by adding a layer of polymer, or other biomolecules.

10. The method contains, according to claim 7, analyte and ligand can be any atoms, ions, molecules, proteins, antibodies and antigens, biological cells or organelles.
